(19) United States
(12) Patent Application Publication
(10) Pub. No.: US 2004/0181115 A1
(43) Pub. Date: Sep. 16, 2004

(54) Method and apparatus for treatment of patients

(76) Inventors: Reuven Sandyk, Roslyn, NY (US); Zvi Kamil, Tel Aviv (IL)

Correspondence Address: Ostrager Chong & Flaherty, 30th Floor, 825 Third Avenue, New York, NY, 10022-7519, US

(21) Appl. No.: 10/486,788

(22) PCT Filed: Aug. 20, 2002
(86) PCT No.: PCT/US02/26387

Related U.S. Application Data
(60) Provisional application No. 60/313,530, filed on Aug. 20, 2001.

(51) Int. Cl. A61N 1/00; A61N 2/02

(52) U.S. Cl. 600/9; 600/15; 607/45; 424/9.2; 600/595; 128/904

(57) ABSTRACT

A system and a method for automated setting of radiation treatment devices using a master computer. The method allows the physician to digitally control the patient's treatment from a master computer and make rapid and fine adjustments in multiple treatment devices. The multiple treatment devices may be situated at the doctor's office, in which case they are connected to the master computer via a local area network (LAN). Alternatively, a network may be used (e.g., the public telephone switching network, an intranet or the Internet) to enable the physician to send radiation therapy from a central location (e.g., a doctor's office) to PC computers or treatment devices at remote sites (e.g., patients' residences). In the former case, the treatment parameters and protocol are then loaded into the radiation treatment device from the remote PC computer, allowing the patient to receive radiation at home or any other preferred location at his/her convenience.

### RELATED PATENT APPLICATION

This application claims the benefit, under Title 35, United States Code, § 119(e), of U.S. Provisional Application No. 60/313,530 filed on Aug. 20, 2001.

### BACKGROUND OF THE INVENTION

This invention relates to the radiation treatment of patients having treatable medical conditions.

U.S. Pat. No. 5,470,846 to Reuven Sandyk (the '846 patent) discloses a method for treating patients for neurological and mental disorders that are associated with and/or related pathogenetically to deficient serotonin neurotransmission and impaired pineal melatonin functions in humans. The treatment comprises the steps of administering a chemical composition that increases serotonin transmission to the patient to be treated followed by the application to the brain of the patient of electromagnetic radiation having an intensity and a frequency appropriate for treating the disorder.

The '846 patent discloses that it was known in the prior art that pulsed magnetic fields in the picotesla intensity range, when applied externally over the head of the patient, are beneficial in the treatment of several neurological disorders including epilepsy, Parkinson's disease, dystonia, tardive dyskinesia, migraine, and multiple sclerosis. See, for example, Anninos et al., (1991) “Magnetic stimulation in the treatment of partial seizures.” International Journal of Neuroscience, 60, 141-171; Sandyk and Anninos (1992) “Attenuation of epilepsy with application of external magnetic fields: a case report.” International Journal of Neuroscience, 66, 75-85; Sandyk (1992) “The influence of the pineal gland on migraine and cluster headaches and the effects of treatment with picotesla magnetic fields.” International Journal of Neuroscience, 67, 145-171; Sandyk (1992) “Weak magnetic fields as a novel therapeutic modality in Parkinson's disease.” International Journal of Neuroscience, 66, 1-15; Sandyk (1992). “Successful treatment of multiple sclerosis with magnetic fields.” International Journal of Neuroscience, 66, 237-250; Sandyk and lacono (1993) “Resolution of longstanding symptoms of multiple sclerosis by application of picotesla range magnetic fields.” International Journal of Neuroscience, 70, 255-269; Sandyk and lacono (1993) “Reversal of visual neglect in Parkinson's disease by treatment with picotesla range magnetic fields.” International Journal of Neuroscience, 73, 93-107); Sandyk (1994) “Parkinsonian micrographia reversed by treatment with weak electromagnetic fields.” International Journal of Neuroscience, 81, 83-93; Sandyk (1994) “Improvement in short-term visual memory by weak electromagnetic fields in Parkinson's disease.” International Journal of Neuroscience, 81, 67-82; Sandyk (1994) “A drug naive Parkinsonian patient successfully treated with weak electromagnetic fields.” International Journal of Neuroscience, 79, 99-110; Sandyk (1994) “Alzheimer's disease: improvement of visual memory and visuoconstructive performance by treatment with picotesla range magnetic fields.” International Journal of Neuroscience, 76, 185-225; Sandyk and Dann (1994) “Weak electromagnetic fields attenuate tremor in multiple sclerosis.” International Journal of Neuroscience, 79, 199-212; Sandyk (1994) “Improvement in word-fluency performance in patients with multiple sclerosis by electromagnetic fields.” International Journal of Neuroscience, 79, 75-90; Sandyk (1994) “Reversal of visuospatial hemi-inattention in patients with chronic progressive multiple sclerosis by treatment with weak electromagnetic fields.” International Journal of Neuroscience, 79, 169-184. The '846 patent discloses that the treatment of such disorders using externally applied magnetic fields can be enhanced by first administering a pharmacological-nutritional composition.

The '846 patent discloses that the magnetic fields are applied to the patient's brain through a transducer (e.g., an array of coils) placed over the scalp. Upon energization of the coils with electric current, the coils produce magnetic fields that are directed into the brain, and particularly in the area of the pineal gland of the patient. Electric current is applied to the coils by a driver comprising a voltage generator and an output resistor, by which the generator is coupled to the coils. Also included in the driver is a timer for activating the generator to provide a sequence of pulses of output voltage that are applied to the resistor for a certain period of time. The voltage generator in combination with the resistor acts as a current source to provide a current to the transducer proportional to the voltage outputted by the generator. The intensity of the magnetic fields produced by the current in the coils is proportional to the magnitude of the current. The voltage generator provides a voltage with a periodic waveform. The generator includes controls for selecting the AC frequency of the voltage, the waveform of the voltage, and the amplitude of the voltage. The '846 patent states, by way of example, that the voltage may be a steady DC voltage, or may be varied with a frequency in the range of 0.1 Hz to 10,000 Hz. The waveform may be sinusoidal, triangular, trapezoidal, square, some other suitable shape, or a combination of more than one of these waveforms.

In the preferred embodiment disclosed in the '846 patent, the transducer comprises a substrate which supports the coils in their respective positions in a two-dimensional array. In one disclosed example, the transducer array has a total of 16 coils arranged in four rows, each row having four coils. Typically each coil has four or five turns, and has a diameter of approximately two centimeters, with an area of approximately three square centimeters. In another disclosed example, the transducer array has a total of 24 coils arranged in four rows, each row having six coils. A cover layer is disposed on top of the substrate and the coils. The substrate and cover layer are formed of a flexible electrically insulating plastic material that permits flexing of the transducer to conform to the curvature of the patient's head. The coils are formed of a flexible electrically conductive material, such as copper, which permits flexing of the transducer.

In the case of energization of the coils with a sinusoidal current, the voltage generator of the '846 patent is operated to output a peak voltage, typically, of four volts relative to ground. This voltage provides a peak current of eight microamperes, which is more than enough current to provide a peak magnetic field intensity of 100 picotesla. The output voltage of the generator is adjusted to provide a desired intensity to the resultant alternating magnetic fields. If desired, the resistance of the resistor may be reduced to provide still larger values of current for greater intensity of the magnetic fields. Upon energization of the coils with electric current, the resultant magnetic fields have lines of force parallel to the axes of the respective coils. The coils are disposed so that the resultant magnetic fields are uniform. The driver and the transducer are capable of providing alternating magnetic fields in a frequency range of 0.1 Hz to 10 kHz, and intensity up to 100 microtesla. Typically, however, the intensity of the alternating magnetic fields is in the range of 6.5-75 picotesla, and the frequency is in the range of 0.5 to 20 Hz.

In accordance with the current treatment protocol, a practicing physician will maintain a plurality of radiation treatment devices at his office. Each radiation treatment device must be individually set or adjusted by the physician or by a technician under the supervision of the physician. The adjustments of the individual features on the driver (i.e., frequency, waveform, amplitude, etc.) are made manually using dials. This is inconvenient for the physician, requiring him/her to be available to administer the treatment at the specific site where the treatment device is located. The need to individually adjust each treatment device manually also effectively limits the number of patients that can be treated simultaneously. Presently, the radiation treatment is limited to a small number of patients in the doctor's office due to restrictions in the amount of space available and the ability of the physician to individually set multiple radiation treatment devices.

In addition, the prior art system employs an analog device that is subject to deviations of the signal parameters during the treatment of a patient. Because the prior art system lacks precise control and fine resolution of the frequency and amplitude of the electromagnetic field signal applied to the patient's head, the physician's control of the patient's symptoms is less than optimal. The previous analog device was inaccurate because: (a) frequency and amplitude were set by hand using knobs; (b) it lacked the fine resolvability of the frequency and amplitude provided by a computerized digital system; (c) most importantly, both the frequency and amplitude could not be maintained stable during treatment with the analog device. Deviations from the desired frequency and amplitude occurred during treatment with this analog device either due to fluctuations in the power of the battery (9-volt battery) or factors inherent to the analog device.

Another practical disadvantage of using electromagnetic fields to treat patients having neurological or mental disorders has been that the patient must come to the physician's office for treatment. Since the treatment does not constitute a cure of the disease, and thus must be repeated two or three times a week (maintenance therapy), it places a major burden on the patient to travel to the doctor's office at specific hours. This is of particular significance for patients who come to see the administering physician from out of state or from overseas. Such patients tend to miss treatments, which negatively affects the success of the procedure. However, it would not be advisable to provide a patient with a radiation-generating therapy device to take home and self-administer the therapy without medical supervision, since many of the Parkinson's patients are elderly individuals who might inadvertently set the wrong treatment parameters on the dials of the driver and experience deleterious side effects.

Thus, there is a general need for a way in which patients having diseases that are treatable by radiation can receive such treatment at home. There is also a need for a device for treating patients with radiation that is portable, programmable and easily operated, e.g., by simple operation of a pushbutton. This would enable a patient to treat him/herself at home by pushbutton operation of a preprogrammed box.

There is also a need for a method and a system for automating the setting of radiation treatment devices, thereby enabling a physician to practice more efficiently and treat more patients. The method should allow the physician to control the patient's treatment from a master computer and make rapid adjustments. In particular, such method and system should enable the physician to send radiation therapy parameters from a central location (e.g., a doctor's office) to remote sites (e.g., patients' residences), allowing the patient to receive radiation treatment at home or any other preferred location at his/her convenience. Such a system would eliminate the need for the patients to travel to the doctor's office for each treatment. Such a method could be used to ensure that patients do not miss treatments, since missed treatments may lead to deterioration in the patient's condition. All patient data, including the parameters for each treatment administered, should be centrally maintained, e.g., in a central database. Preferably the remote treatment method provides for the feedback of treatment information from remote sites to the central office. Such a method and system will permit a single physician to treat a large number of patients simultaneously without regard to the locations of the patients.

In addition, there is a need for a method and a system for automatically setting a radiation treatment device by means of digital control signals. The use of a computerized digital system would allow a physician to set the electromagnetic signal parameters required for treatment. A digitally controlled radiation treatment device would enable a physician to refine the treatment parameters to optimize the therapeutic effect on the specific symptoms of each patient.

As used herein, the term “radiation” means the emission of waves or particles from a source and the propagation of these waves or particles through a medium. Thus the term “radiation”, when used herein without the modifier “electromagnetic”, is not limited to electromagnetic radiation.

### BRIEF DESCRIPTION OF THE INVENTION

The present invention, in its broadest scope, is directed to a system and a method for setting radiation treatment devices using a master computer. Patient-specific digital data representing waveform parameters and treatment protocol can be loaded into each radiation treatment device. The use of digital data enables the physician to “fine tune” the treatment to achieve an optimal response of the patient's symptom.

The method in accordance with one embodiment allows the physician to control the patient's treatment from a master computer and make rapid adjustments in multiple treatment devices. The multiple treatment devices may be situated at the doctor's office, in which case they are connected to the master computer via a local area network (LAN). Alternatively, a network may be used (e.g., the public telephone switching network, an intranet or the Internet) to enable the physician to send radiation therapy parameters from a central location (e.g., a doctor's office) to remote sites (e.g., patients' residences), allowing the patient to receive radiation at home or any other preferred location at his/her convenience. Such a system would eliminate the need for the patients to travel to the doctor's office for each treatment. Such a method could be used to ensure that patients do not miss treatments, since missed treatments may lead to deterioration in the patient's condition. All patient data, including the parameters for each treatment administered, should be centrally maintained, e.g., in a central database. Such a method and system will permit a single physician to treat a large number of patients simultaneously without regard to the locations of the patients.

The preferred range of electromagnetic flux density applied to the patient is 1 to 100 picotesla, with the optimal mean range being 6.5-10 picotesla. The preferred frequency range for the electromagnetic signal is 0.5 to 20 Hz.

One aspect of the invention is a method of treating patients diagnosed as having a neurological disorder, comprising the following steps: (a) programming a computer to output digital data representing parameters for electromagnetic radiation, the parameters being designed to effect treatment of a neurological disorder; (b) coupling a radiation treatment device to the computer for receiving the digital data output; (c) coupling an electromagnetic radiation transmitter to the radiation treatment device; (d) positioning the electromagnetic radiation transmitter so that transmitted electromagnetic radiation enters the brain of a patient diagnosed as having the neurological disorder; and (e) activating the electromagnetic radiation transmitter to transmit electromagnetic radiation as a function of at least digital data output.

Another aspect of the invention is a method of treating patients at sites remote from a central office, comprising the following steps: (a) generating first through N-th digital data sets comprising waveform parameters and treatment protocol data, wherein N is a positive integer greater than unity; (b) sending the first through N-th digital data sets from a central office to respective first through N-th remote locations; and (c) treating respective first through N-th patients at the first through N-th remote locations, each treatment being a function of the respective one of the first through N-th digital data sets received at the respective remote location.

A further aspect of the invention is a system for treating patients having Parkinson's disease or other neurological disorder, comprising: a radiation treatment device comprising an antenna and circuitry for converting a digital data set into analog drive signals that are output to the antenna; and a computer programmed to send the digital data set to the radiation treatment device via an electrical pathway.

Yet another aspect of the invention is a radiation treatment device comprising: an antenna; a controller programmed to output digital waveform signals that are a function of stored waveform parameters and treatment protocol data; and a digital-to-analog converter for converting the digital waveform signals into analog waveform signals, wherein the antenna is driven by drive signals derived from the analog waveform signals.

Another aspect of the invention is a system for treating patients at sites remote from a central office, comprising: a central treatment management computer programmed to generate first through N-th digital data sets comprising waveform parameters and treatment protocol data, wherein N is a positive integer greater than unity; means for sending the first through N-th digital data sets from the central treatment management computer to respective first through N-th remote locations; and means for treating respective first through N-th patients at the first through N-th remote locations, each treatment being a function of the respective one of the first through N-th digital data sets received at the respective remote location.

A further aspect of the invention is a method of treating patients having a neurological disorder, comprising the following steps: (a) determining a digital data set that characterizes a radiation treatment for a patient having a neurological disorder; (b) loading that digital data set into a radiation transmission controller; and (c) transmitting radiation into a patient's brain under the control of the radiation transmission controller, the transmitted radiation having properties that are a function of the loaded digital data set.

Yet another aspect of the invention is a computer programmed with parameter selection software, the parameter selection software comprising an operator interface for operator selection of parameters characterizing radiation for treating a patient diagnosed to have a neurological disorder and operator input of a load parameter command, and further comprising a routine for outputting a message comprising digital data representing the selected parameters in response to operator input of a load parameter command.

Other aspects of the invention are disclosed and claimed below.

### BRIEF DESCRIPTION OF THE DRAWINGS

FIG. 1 is a block diagram depicting a system for managing treatment of patients from a master computer in accordance with one embodiment of the present invention.

FIG. 2 is a diagram showing the circuitry of a radiation treatment device that can be incorporated in the system shown in FIG. 1 in accordance with one implementation.

FIG. 3 is a block diagram depicting a system for managing remote treatment of patients over the Internet in accordance with another embodiment of the invention. The radiation treatment device shown in FIG. 2 can also be incorporated in the system shown in FIG. 3.

### DETAILED DESCRIPTION OF THE INVENTION

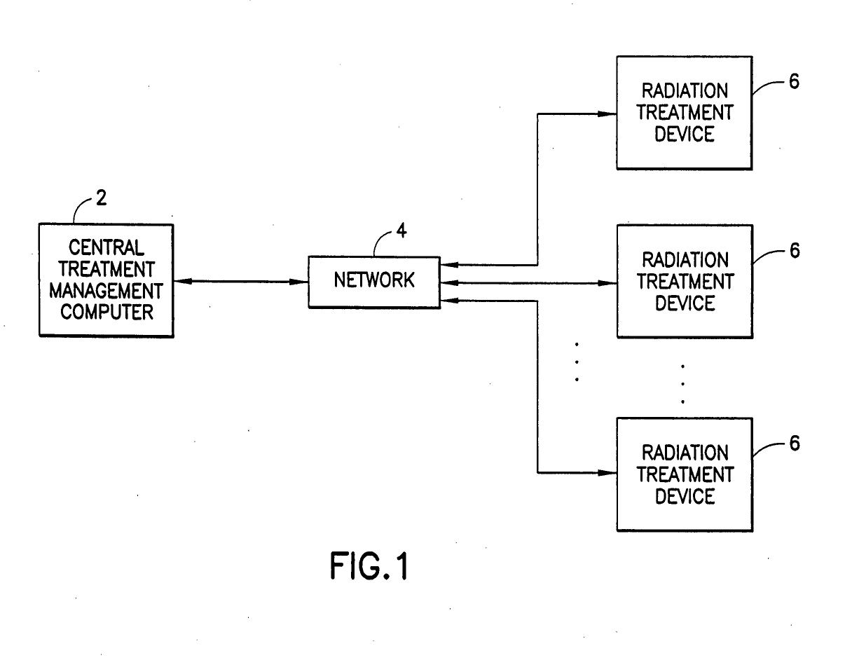

Referring to FIG. 1, the present invention is directed in part to a system and a method for automating the setting of radiation treatment devices 6 using a central treatment management computer 2. In accordance with some embodiments of the invention, the computer 2 communicates with the radiation treatment devices 6 via a network 4. Alternatively, the central computer may communicate with a plurality of devices via respective line simulators. In accordance with a further alternative, the radiation treatment device may be directly connected to the central treatment management computer. In general, the radiation treatment device is loaded with waveform parameters and treatment protocol data by the central computer. Optionally, the radiation treatment can be activated from the central computer, although the radiation treatment device is provided with a manual input means, e.g., a pushbutton, for activating the radiation treatment.

The radiation treatment devices 6 may be located in a doctor's office, in the same building, in different buildings at a hospital facility, or at different remote sites (e.g., at the residences of patients to be treated). The network 4 may comprise an LAN, an intranet, the Internet, the public telephone switching network, a wireless communications network, or any other communications network that would enable a computer to communicate with a selected one of a plurality of radiation treatment devices. The central treatment management computer may comprise a personal computer, a server, a laptop computer, a hand-held computer or any other computer programmed to manage multiple radiation treatment devices and interface with the network 4.

For example, the radiation treatment devices 6 may all be located in a doctor's office and connected to a master computer 2 via an LAN. Alternatively, the radiation treatment devices 6 could be located in different states and different countries, at the residences of patients to be treated. Such far-flung radiation treatment devices 6 would preferably be connected to a master computer 2 located at a doctor's office via the public telephone switching network or via the Internet.

In accordance with those embodiments fitting the general architecture shown in FIG. 1, each radiation treatment device 6 is used to treat an individual patient in accordance with an individualized or patient-specific treatment protocol. The treatment protocol for each patient is preferably stored in a database associated with or incorporated in the central treatment management computer 2. In particular, the patient database may be stored on the hard disk of the central treatment management computer 2 or on a separate database server which communicates with computer 2 via network 4 or a different network. Preferably, the patient database includes the history of past radiation treatments and the protocol to be used in the next treatment for each patient. The history of past radiation treatments preferably takes the form of a chronological file which is continually updated after completion of each treatment the parameters of the protocol for the next treatment are settable or adjustable by the physician or other authorized person via a graphical user interface comprising a respective display field for each parameter. In the instance where the patient is to be treated with electromagnetic fields, the settable parameters include the frequency, amplitude and waveform as well as the duration of the treatment or each phase of the treatment, i.e., any parameter may be varied in different phases of one treatment in accordance with a designed treatment protocol. This information is stored digitally in computer memory, ready to be delivered to a radiation treatment device upon request.

In accordance with one embodiment wherein a plurality of radiation treatment devices 6 are located in a doctor's office, it is preferred that the central treatment management computer 2 be used by the physician to load a treatment protocol in the memory of a particular radiation treatment device and then activate that device after the radiation treatment device has been applied to the patient. All commands and treatment parameters are sent as digital information over the LAN (or line simulator) connecting the radiation treatment devices to the master computer. When the treatment duration has expired, the radiation treatment device would then automatically deactivate itself.

In accordance with another embodiment wherein a plurality of radiation treatment devices 6 are located at remote sites, a patient at a remote site can use the radiation treatment device to call the central treatment management computer 2 and download waveform parameters and a treatment protocol to a particular radiation treatment device under his/her control. Later the patient at the remote site can activate the radiation. When the treatment duration has expired, the radiation treatment device 6 would then automatically deactivate itself in accordance with the stored protocol. This enables a patient to activate the radiation treatment device and initiate the programmed radiation treatment at a time that is convenient for the patient. Furthermore, the patient could even receive signals representing multiple treatment protocols, to be delivered on different dates or at different times during the same day, during one call to the central computer. Since the radiation treatment device preferably has its own microcontroller and memory, multiple individual treatment protocols can be stored in memory and then read individually from memory by the microcontroller at different times. The ability to store and selectively recall treatment protocols allows the greatest freedom and flexibility for both the physician and the patient. In particular, the patient is free to choose when to load a treatment protocol and when to initiate the programmed radiation treatment.

To prevent the loading of treatment parameters into an incorrect or mistaken remotely located radiation treatment device, the latter is preferably programmed to accept a treatment protocol only if a header in the transmission from the central computer contains an identification code or personal identification number (PIN) that is unique to that particular radiation treatment device. The radiation treatment device will not accept the treatment protocol data, i.e., will not be activatable, unless the treatment device has validated the ID code or PIN received from the central computer as part of the treatment protocol transmission.

The remote radiation treatment device may also be programmed to record the time, duration and parameters of each treatment in a treatment history file. This treatment history file can be accessed via the RS232C channel by a computer. Alternatively, the radiation treatment device can be programmed to periodically upload the treatment history file to a central computer via a telephone line. The radiation treatment device may be provided with both a permanent treatment history file which is unaffected by uploading and an updated treatment history file that is uploaded and then erased in response to a message from the central computer acknowledging reception of the file.

Alternatively, a PC computer connected to the radiation treatment device at a remote site can be programmed to request the treatment history file from the associated radiation treatment device in response to the input, via an operator interface of the PC computer, of an appropriate instruction by the patient. The patient could in turn initiate this process in response to receipt of an e-mail from a central office requesting that the treatment history file be uploaded.

As used herein, the term “treatment protocol” means the application of radiation during one or more time intervals over the course of a treatment session. In other words, a single treatment session may comprise two or more separate radiation treatments applied at prescribed intervals, such as the time intervals disclosed in U.S. Pat. No. 5,691,324 to Reuven Sandyk.

The medical radiation treatment device disclosed herein outputs analog electrical signals to a transmitting coil in accordance with waveform parameters and treatment protocol data preloaded into the treatment device. A specific set of waveform parameters and treatment protocol data are selected by a physician for each patient to be treated. In the case where the radiation treatment device will be used to treat successive patients, for example, in the physician's office, the device must be re-programmed, i.e., the specific set of waveform parameters and treatment protocol data must be pre-loaded into the device prior to each treatment. Moreover, the initial set of waveform parameters and treatment protocol data input to the device for a particular patient may prove to be less than optimal, in which case the physician may adjust or “fine tune” the settings to enhance the control of the patient's symptoms. The adjustment procedure may be repeated until the settings are optimized.

Medical treatment administered using a prototype of the device disclosed herein (which was connected via an RS232C serial connection to a personal computer) has shown that having a digitally-controlled treatment device with appropriate resolution capability has shown superior clinical results. The digital computer-regulated device allows a significantly more precise control and resolution capability of the frequency (0.01 Hz deviation) and amplitude (0.01 volt) selected for the electromagnetic field signal applied to the patient's brain. In contrast to the previously used analog treatment device mentioned in the Background of the Invention section, which was subject to deviations of the signal parameters during the treatment, the system disclosed herein, by allowing a more precise “fine tuning” of the electromagnetic signal administered to the patient, enables the treating physician to provide a more optimal control of the patient's symptoms. This is a marked improvement over the prior art. With the new computerized system, a magnetic field as low as 1 picotesla has been used in the treatment of Parkinson's disease patients. The preferred range of flux density applied to the patient is 1 to 100 picotesla, with the optimal mean range being 6.5-10 picotesla.

In the case of patients with Parkinson's disease, the core symptoms of the disease, such as tremor, rigidity, bradykinesia, postural instability and speech impairment, can be targeted more precisely, thus producing a greater degree of symptomatic improvement. In addition, by targeting the core symptoms of the disease with a more precise electromagnetic signal, the treating physician is able to achieve the following: (1) reduce the duration of magnetic treatment (by up to 50% in some patients) without compromising its efficacy; and (2) prolong the beneficial effect of each treatment (in some patients by up to 100%), thus keeping the Parkinsonian patient functional for a longer period of time before he/she is due for repeat treatment.

In patients with Parkinson's disease, the optimization of the proper frequency and amplitude of the electromagnetic signal was achieved during the treatment on the basis of clinical criteria by monitoring the patient's hand tremor, muscular rigidity (cogwheel rigidity in the wrists), manual dexterity (finger tapping) and speech impairment (repetition of the syllable la, la, la). For example, in patients who exhibit tremor in the upper limbs, the calibration of the optimal frequency and amplitude was directed at the point of maximal suppression of the hand tremor.

The medical radiation treatment device in accordance with one embodiment of the invention has three modes of operation. The three operating mode are as follows: (1) PC Computer mode; (2) Treatment Only mode; and (3) Telephone Line mode.

In the PC Computer mode, the radiation treatment device is connected to a PC computer, either at a central location, such as a physician's office or other patient treatment facility, or at a remote site, such as the patient's home. In either case, the radiation treatment device is preferably connected to the PC computer via an RS232C serial communications channel. However, the present invention is not limited to any particular type of communications channel. Regardless of how the radiation treatment device receives waveform parameters and protocol data, the device is programmed to transmit radiation in accordance with received waveform parameters and protocol data when activated.

In the case where the radiation treatment device is set up at a physician's office or other patient treatment facility, the device may be one of a multiplicity of devices connected to an RS232C network. A console-like PC computer, i.e., the aforementioned “central treatment management computer”, is programmed with software for sending control data to the radiation treatment devices via the RS232C network. Alternatively, a single radiation treatment device could be connected to the central treatment management computer directly, without an intervening network, by connecting an RS232C port on the treatment device to an RS232C port on the central treatment management computer.

Each radiation treatment device can be connected to the central treatment management computer (directly or via a network) on one side and to the patient on the other side. The system operator controls (by means of the console) the different parameters for adjusting the operation of each treatment device to the specific needs of each patient being treated. The system sets the drive signal waveform parameters, including frequency, amplitude and shape of the waveform, and the treatment protocol data, including duration of the treatment, the number of treatment cycles to be administered, the duration of intervening rest periods, and generally the whole protocol of the treatment procedure. Applying the drive signal waveform parameters, the treatment device will drive the coils on the patient's head to transmit magnetic field signals in accordance with the treatment protocol.

In accordance with a further embodiment of the invention, the central computer is programmed with parameter selection software to facilitate the automated setting of the treatment devices. The parameter selection software comprises an operator interface for operator selection of parameters characterizing radiation for treating a specific patient diagnosed to have a neurological disorder and operator input of a load parameter command for loading the parameters into the treatment device. The parameter selection software further comprises a routine for outputting a message comprising digital data representing the selected parameters in response to the operator input of a load parameter command, which can be a click on a virtual Send button on the display screen. Likewise the screen has a virtual Save button for saving the selected parameters to computer memory. The radiation treatment device comprises a microcontroller coupled to the central computer via a cable or network. The microcontroller is programmed to output digital sample values representing the desired radiation. These digital sample values are a function of the digital data loaded by the central computer. The system further comprises an assembly for transmitting radiation that is a function of the digital sample values. The assembly comprises a head coil antenna designed to transmit radiation into a patient's brain. In accordance with one embodiment, the head coil antenna comprises a copper wire wound in the shape of a numeral 8 in multiple windings (e.g., five). This 8-shaped multi-turn coil is placed on top of the patients head with the bottom of the 8-shape to the front and the top of the 8-shape to the rear of the head.

Preferably, the operator interface comprises a graphical user interface, which in turn comprises respective windows for selecting an amplitude, a frequency, a waveshape, a treatment duration, etc. These windows may be displayed concurrently or in sequence on said display screen. The physician can select the desired parameters by the simple expedient of clicking on a mouse. The software will incorporate the selected parameters into a formatted message, which is sent to the treatment device. The treatment device in turn is programmed to recognize each field in the message and treat each parameter correctly.

In accordance with other embodiments, a patient can download waveform parameters and treatment protocol data from a website to a PC computer via the Internet. Then the patient can use the PC computer to load the same digital data into the radiation treatment device. In this case the PC computer at the remote site is operated by the patient, and the physician participates, if at all, only via a telephone line or via Internet connection, by means of which the physician may effectively control or monitor the ongoing treatment and download adjusted waveform parameters and treatment protocol data to the remote PC computer. Thus, the physician may monitor and adjust the treatment remotely. The loaded treatment device is then activated by a person, e.g., the patient, at the remote site.

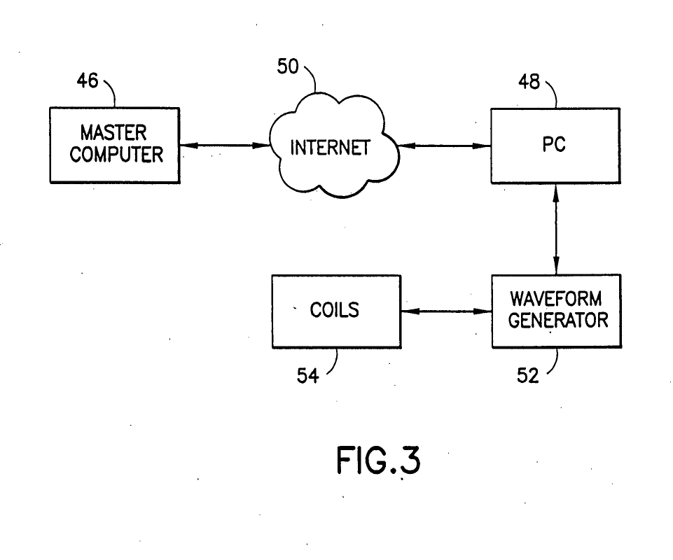

In the foregoing case there is an option wherein the physician or operator at a central location may talk and see the patient during treatment at a remote site by using up-to-date video/Internet technology to see and speak to the patient. This requires that a video camera be coupled to the PC computer and aimed at the patient. This enables the physician to observe the patient while the treatment is ongoing. At the remote site, the radiation treatment device can be connected via its RS232C connector to the RS232C connector on a standard PC. The PC modem is then connected to the telephone line (or by other means), using it for either direct dialing, modem to modem, to a central treatment management computer or using the Internet to reach the central treatment management computer, as shown in FIG. 3.

In the PC Computer mode, the radiation treatment device may be activated by a Start command input via the RS232C channel, e.g., from a computer at the central office or at the remote site, or it may be activated manually by pressing an “On” pushbutton.

In the Treatment Only mode, the radiation treatment device functions as a standalone device after it has been programmed or loaded with waveform parameters and treatment protocol data for a particular patient. When the device is fully loaded, it can be transported anywhere for selective activation at the convenience of the patient, for example, at the patient's residence, in a hotel room, etc. In this mode, the device is operated by command of the patient, assistance or an aide. Upon being activated, the device performs the preloaded treatment protocol.

In the Telephone Line mode, a radiation treatment device at a remote site can be programmed or loaded by downloading digital data from the physician's clinic or other central patent treatment facility via the public telephone switching network. In this mode, the radiation treatment device can connect to a telephone line and make a call to the central facility. During this connection, the radiation treatment device can receive programming for performing a predetermined number of treatments, each treatment being activatable by the patient at a time chosen by the patient. When the preset number of treatments has been exhausted, the radiation treatment device will enter a state wherein it cannot be activated by the patient until it has been, in effect, “recharged” with additional treatments. Any conventional security means may be provided to prevent unauthorized or unapproved activation of the radiation treatment device by the patient. For example, the central source of the programming can be required to input a password or special code (known only by the attending physician or central administrator) in the header of the message sent to the radiation treatment device before the latter will allow itself to be programmed or loaded with more treatments.

In order to selectively operate in any one of the above-described modes, the radiation treatment device in accordance with one embodiment of the invention is constructed with three parts: (1) a communications interface for communicating with a computer via a serial communications channel; (2) a communications interface for communicating with a computer via a telephone line; and (3) a central unit that transmits radiation as a function of the preloaded waveform parameters and treatment protocol data. Optionally, parts (1) and (2) may be combined into a single communications channel. In accordance with an alternative embodiment, the radiation treatment device can be further provided with an operator interface, a display screen and Internet capability, e.g., web browser software, with the patient being able to download, via a standard communication line, waveform parameters and treatment protocol data from a web site.

One aspect of the invention is the capability to treat patients at locations remote from a central office. This aspect of the invention is not limited to radiation treatment. Such a radiation treatment device comprises a central microprocessor or digital microcontroller having peripheral devices, such as D/A converters and other electronic components like LEDs, transistors, amplifiers etc., as needed to perform the treatment task. In the general case the microcontroller can also control small motors and/or laser devices as needed for the patient's treatment. In the disclosed embodiment, the treatment device transmits low-intensity electromagnetic fields for the treatment of neurological disorders.

In order that a device of this kind will be suitable for performing the radiation treatment, it should be built using digital technology so that the timing is controlled by a crystal oscillator and the amplitudes are generated by D/A converters, digital potentiometers, etc. so that selecting a setup can be repeated at the same instrument multiple times and for long periods of time (in the limits of the selected precision). Also, all instruments may be set up to the same values to ensure that each device has the same performance (up to the selected precision) and to ensure that each patient receives the correct treatment independent of the specific device used.

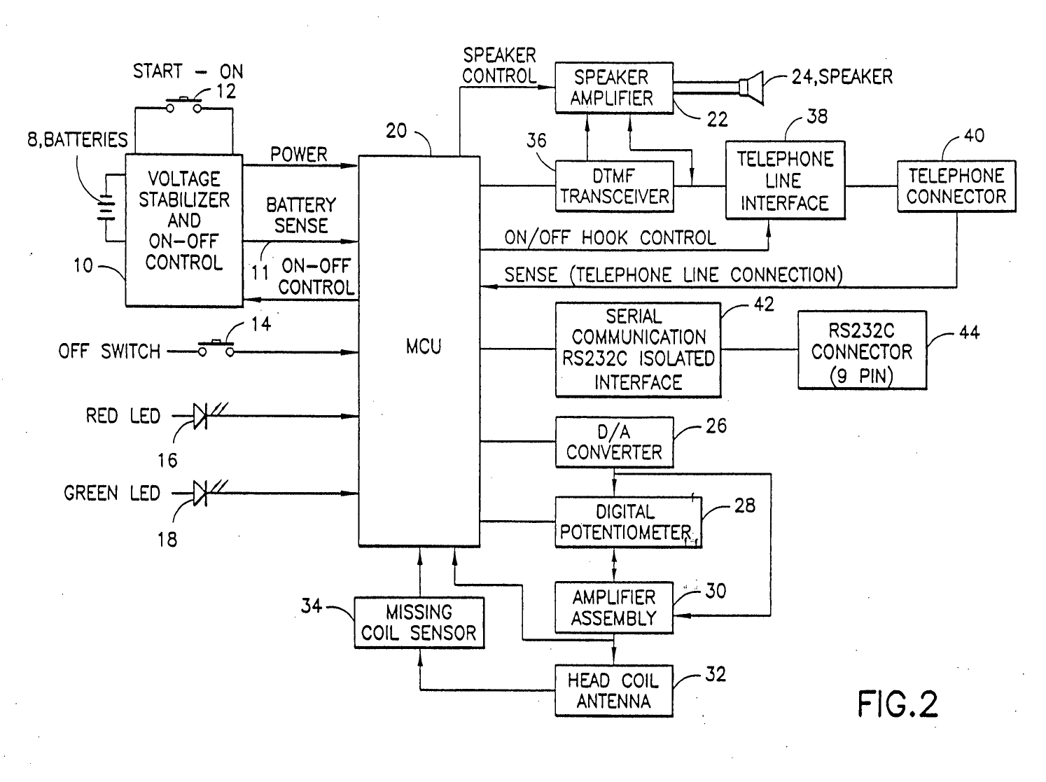

FIG. 2 shows the circuitry of a battery-powered radiation treatment device in accordance with one embodiment of the invention. The radiation treatment device comprises a microcontroller 20 having a port for coupling the radiation treatment device to a telephone line via a DTMF transceiver 36, a telephone line interface 38 and a telephone connector 40. Alternatively, a small digital processor other than a microcontroller, e.g., a microcomputer, can be used. The microcontroller 20 incorporates non-volatile memory (e.g., battery-powered memory, flash memory or other non-volatile memory technology) for storing also waveform/protocol parameters and other data received from the master computer via the telephone line. Such waveform/protocol parameters may include some or all of the following: gain, amplitude, frequency, waveshape, duration of treatment, time of treatment, number of times a treatment may be repeated, and other relevant functions, such as amplitude modulation, frequency modulation and phase modulation.

As an alternative to communication via a telephone line, the radiation treatment device also comprises an RS232C communications channel by means of which waveform parameters and treatment protocol data can be loaded into the radiation treatment device from a PC computer, located either at the remote site or at a central treatment facility, as previously described. The channel comprises serial communication RS232C isolated interface 42 and an RS232C 9-pin connector 44.

When the device is turned on, it checks its environment by putting the telephone connection into an “off-hook” state (closing the loop). If the telephone line is connected then this will be sensed by the microcontroller 20 (detecting the “loop current”). In this case the microcontroller 20, via the DTMF transceiver 36, will automatically dial to the central treatment management computer and will communicate to perform the needed task. Upon termination, the microcontroller 20 will turn itself off. If the telephone line is not connected, then the radiation treatment device will monitor the RS232C channel. If any RS232C activity is detected, the device will put itself into the PC Control mode. If the telephone line is not connected and the RS232C channel is inactive, then the device will enter the Treatment Only mode, e.g., by starting a treatment procedure used its last updated stored treatment protocol.

The microcontroller 20 processes the loaded treatment parameters and outputs a digital signal representing a waveform having a desired frequency and shape for driving the coil of antenna 32, which is placed on the head of a patient being treated. A digital-to-analog (D/A) converter 26 converts the digital signals output by the microcontroller 20 into an analog signal having the desired frequency and waveshape. The microcontroller 20 also outputs a digital value representing a setting to a digital potentiometer 28. The function of the digital potentiometer 28 is to adjust the level of the treatment signal, since the D/A converter 26 is always giving full amplitude. The output of the D/A converter 26 and the digital potentiometer 28 form the input signal to the amplifier assembly 30, the output of which is the current applied to the head coil antenna 32.

A missing coil sensor 34 provides a signal to the microcontroller 20 when the head coil antenna 32 is defective, disconnected or improperly connected to the port (on the housing of the radiation treatment device) in which the cable to the head coil antenna is plugged. In response to receipt of a detection signal indicating disconnection or improper connection of the head antenna, the microcontroller 20 will output an alarm control signal to a speaker amplifier 22, causing an alarm signal to be produced by a speaker 24. The microcontroller 20 will turn off the power supply after the alarm signal has been issued.

The microcontroller 20 outputs the digital waveform signals in accordance with the stored treatment protocol data. For example, the treatment protocol may comprise a single continuous treatment or a plurality of treatment cycles separated by quiescent intervals or rest periods. The number of available treatments, which is also loaded along with the waveform parameters and the treatment protocol data, is stored in the memory of the microcontroller and reduced by unity each time a treatment is completed. When the number of available treatments equals zero, the remote treatment device is programmed to refuse to be activated and to issue a signal that no further treatment is allowed. In addition, a voice message can be issued via the speaker 24, stating that further treatments are not authorized and instructing the patient to contact his physician or clinic.

Still referring to FIG. 2, the microcontroller 20 is powered by a battery or batteries 8. The voltage from the battery is supplied to the microcontroller 20 via a voltage stabilizer/on-off control circuit or chip 10. The voltage supplied by the battery is stabilized by the voltage stabilizer. The on-off control portion of chip 10 receives a control signal from the microcontroller 20. The remote treatment device can turn itself off by command from the microcontroller. For example, the system can be programmed to shut down under the following circumstances: when the treatment has terminated; in response to an illegal operation; and when sensor 34 detects that the head coil antenna 32 is disconnected or improperly connected or defective. Normally most problems regarding the proper operation of MCU 20 or its software will result in turn off. The output of the analog chain (i.e., the D/A converter 26, the digital potentiometer 28 and the amplifier assembly 30) is connected into an A/D input of the MCU 20 to enable autotest of the proper operation of that subsystem. A Start-On pushbutton 12 is provided to turn the system on (after it is shut down). An Off pushbutton 14 is also provided for shutting down the system at any time. More precisely, the microcontroller 20 is programmed to send an Off command to chip 10 in response to pushbutton 14 be depressed. Optionally, the microcontroller can be programmed to take some other action in response to depression of pushbutton 14, in which case the latter could serve as a function switch in certain situations.

Numeral 11 indicates a low-voltage sense circuit that outputs an analog signal proportional to the current battery voltage to an input of the microcontroller 20. The microcontroller incorporates an A/D converter that converts the analog signal to a digital value. That digital value is compared to a stored threshold value. When the battery voltage falls to a level corresponding to the stored threshold value, the microcontroller causes the red LED 16 to blink, indicating that the battery needs to be replaced. The red LED 16 is turned on as long as the radiation treatment device is activated. A green LED 18 is activated whenever the speaker is used and blinks when treatment is being performed. The green LED lights continuously for one minute after the end of treatment whenever number of available treatments remaining is either one or two. The speaker 24 is also connected to the telephone line (not shown in FIG. 2), so when the line is active, the speaker will transmit all audio signals. This enables the user to listen to the dial tone, dialing DTMF, communication and end of transmission. The speaker is used in other modes on start treatment, pause, begin second part of treatment, and end of treatment. The speaker emits (under the control of the microcontroller) an alarm when the head antenna is disconnected, not properly connected or defective and when no more treatments are available. The microcontroller will turn the remote treatment device off after the alarm signal has been issued.

The waveform parameters and treatment protocol data may be fed to the microcontroller 20 via either the telephone line interface or the RS232C interface. Alternative communications channels can be employed. All parameters and protocol data are stored in the central treatment management computer and loaded into the radiation treatment device either directly or via a PC computer connected to the treatment device. The microcontroller 20 can store any desired waveform by receiving a series of values that can be repeatedly transmitted as an amplitude and time interval as selected by data transferred from the master computer. Alternatively, the microcontroller can have an internal algorithm to generate a waveform of the desired shape, amplitude and frequency to be supplied to the head coil antenna.

At the site of the origination of the treatment, e.g., a doctor's office, the radiation treatment device can be connected to the master computer and, as necessary, the computer operator can change all parameters on line in real-time via code loaded in and transmitted from the master computer. Alternatively, each radiation treatment device can be programmed directly by removing the cover of the casing in which the radiation treatment device is housed and directly applying an external programming device (mainly used by the manufacturer). This direct method can be used to change the software of the device or to load waveform parameters and treatment protocol data.

In accordance with the disclosed embodiments, the master computer is loaded with user-friendly software that enables easy generation of waveshape, gain, frequency change and also modulation of the amplitude, frequency or phase of the selected waveform by other waveforms (amplitude and timing) for treating the patient. The waveform parameters can be generated in the computer either by point-to-point design of the waveform or using mathematical functions to generate the data.

The disclosed embodiment is powered by batteries. However, in place of batteries, an appropriate power supply can be used (connected to mains). However, the current consumption of the radiation treatment device is so low that the cheapest and most safe and convenient method is to use batteries.

FIG. 3 depicts a system for remote treatment of patients via the Internet 50. The master computer 46 stores data and treatment parameters for each patient in a database. Each patient at a remote site, e.g., a patient's residence, has a personal computer 48, a waveform generator 52 coupled to the personal computer 48 (e.g., via an RS232C communications channel), and transducer coils 54 connected to the waveform generator 52. The master computer 46 preferably comprises a web server that allows authorized patients to download a predetermined number of treatments (including waveform parameters and treatment protocol data for each treatment, as well as any necessary operating software) to their personal computers 48. Each patient has a separate file stored in a database. The database may reside in the master computer 46 or a separate database server networked (e.g., via an LAN at the doctor's office) to the master computer. After inputting a personal identification number (PIN) and a password, each patient may download a web page displaying personalized patient information, treatment schedule data, treatment parameters and protocol data, and number of times that a treatment may be applied. When displayed as a web page on the personal computer 48, the patient can store this downloaded information. The patient can then disconnect from the master computer 46.

Later, when the time for radiation treatment arrives, the patient will mount a headgear assembly (which incorporates the coils 54 of the previously described head antenna) on his head and then activate a treatment control program on the personal computer 48. The treatment control program retrieves the relevant treatment data and outputs control signals to a waveform generator 52 (e.g., of the type depicted in FIG. 2). The waveform generator then outputs pulses or waves having the desired amplitude, frequency, shape and duration to the coils 54. The coils in turn generate electromagnetic fields that are directed, if the headgear is correctly positioned, toward the target area in the patient's brain. Preferably the treatment control program tracks the number of treatments and will decline to initiate a new treatment if the number of treatments already performed equals the maximum number of treatments authorized or approved by the physician. For example, the PC computer is provided with software that refuses to enable the waveform generator if the patient has downloaded ten treatments and ten treatments have been delivered. Alternatively, a patient may be authorized to receive a predetermined number of treatments during a predetermined time period, this cycle being repeatable for a predetermined number of such time periods, e.g., once per week. The PC computer is provided with software for monitoring the treatments and enabling the waveform generator only in accordance with the stored treatment program.

Alternatively, instead of the patient interacting with a web page, a physician could send a respective treatment protocol file by e-mail to each patient. The patient could then open his e-mail, store the file, and then enable a program that would read the treatment protocol file and set or re-program the waveform generator in accordance with the treatment parameters of the received protocol.

In accordance with a further embodiment, the personal computer 48 has a multiplicity of treatment protocols and/or sets of treatment parameters stored in encrypted or other secure format on a hard disk. The patient must then use a program that is able to decrypt the stored treatment information only if the patient inputs a decryption key in response to a prompt. The patient must obtain that decryption key from the master computer, e.g., by connecting to the master computer and providing a fee, e.g., by charging to a valid credit or debit card. The master computer 46 and the personal computer 48 may both be programmed with a random number generator that is used to generate a unique decryption key for each treatment.

In accordance with a further aspect of the invention, in embodiments where the patient contacts the master computer and requests authorization and parameters for a treatment, the master computer may be programmed with the physician's prescription concerning how many and how frequently the patient should be treated. In cases where a patient request is precluded by the physician's prescription, the master computer is programmed to decline the patient request. The rejection may be accompanied by an appropriate message to the patient, e.g., an instruction for the patient to contact his/her physician. When authorization for a treatment is prestored in a master computer accessible via the Internet, a patient may contact the master computer for a treatment at any time of day.

Thus a physician has the ability to provide treatment to patients on a “per call” basis or to pre-approve multiple treatments that can be downloaded to the patient and performed at the patient's convenience over a prescribed period of time. In the latter case, treatment restrictions can be downloaded as part of the treatment protocol data, with restrictions being enforced by a monitoring program in the patient's PC computer. This monitoring program will maintain a chronological log of all treatments and will enforce treatment restrictions each time a new treatment is requested. These treatment restrictions may include the limitation that only one treatment can be delivered for a given time period, e.g., one treatment per week. Thus, even though the patient has been authorized by the central office to receive more than one treatment, the PC computer at the remote site will allow only those treatments in compliance with the restrictions imposed by the central office.

As previously mentioned, the optimal waveform parameters must be calibrated by the physician based on observation of the response of the patient's symptoms to different sets of parameters. The aim of the treatment method disclosed herein is to achieve optimal response to the electromagnetic signal that is transmitted into the patient's brain. Optimal clinical response occurs when the signal administered to the patient's brain is of a specific frequency, amplitude, and waveshape. The process of calibration attempts to establish the proper frequency, amplitude and waveshape that will induce the optimal clinical response (i.e., maximal suppression of tremor, highest degree of improvement in muscular rigidity and speech, etc.). Parkinson's disease is not a clinically uniform disease and therefore each patient exhibits his/her specific “magnetic imprint”, i.e., specific signal parameters (frequency, amplitude, waveshape) which, when administered properly, will induce the most optimal response.

In order to find out the proper signal parameters for an individual patient, the physician must try different frequencies, amplitudes and waveshapes. The computerized digital system disclosed herein allows such fine calibration with resolution of the frequency of 0.01 Hz and that of the amplitude of 0.01 volt. Such minute, rather trivial changes in frequency and amplitude may have a great impact on the patient's response to treatment. A preferred calibration technique is to use changes in hand tremor to establish the optimal parameters because the tremor is easily quantifiable either by direct inspection of the patient's hand tremor (i.e., amplitude of the hand tremor) during treatment or by having the patient draw continuous circles (i.e., Archimedes circles) or straight lines on a plain sheet of paper. Tremor is manifested by deviations from the straight lines or the circular lines. The desired signal parameters to be used for the treatment are those that result in the greatest degree of rectification of these deviations from a straight or circular line.

Once the patient's parameters have been established during his initial visit to the office or central clinic, these may change over time and periodic recalibration may be necessary. The patient may need to return to the office after using the device at home for a specified period of time and have his parameters recalibrated. Recalibration of the patient's radiation treatment device can be performed remotely through the central computer stationed in the office or central clinic. For example, the physician at the central office could download a set of parameters to the remote device and then instruct the patient, via a separate communication channel (e.g., e-mail or second telephone connection) to administer a treatment and then draw a set of straight lines during that treatment. This procedure can be repeated any number of times, each time using a different set of parameters. At the end of this process, the patient can scan the drawn lines for each procedure and e-mail the scanned images to the physician. The physician can then view the images and determine which set of parameters produced the smallest deviation in the lines, indicating the optimum effect on the patient's hand tremor. These parameters can then be used by the physician to calibrate the remote radiation treatment device. For patients who do not have a scanner and computer with internet connection, these patients can call the physician while being treated and report on the degree of tremor suppression either by direct inspection of their hands or by changes in the drawing of a line or a circle. The physician can also assess the degree of speech improvement on the phone by having the patient repeat the syllable la, la, la.

There is also the possibility that a camera placed in the patient's home could be used to transmit pictures via the Internet so that the patient's response to the new parameters could be seen and calibrated remotely at the central office. This can be accomplished using commercially available equipment, such as a wireless video camera mounted to acquire a video of the patient during treatment; a wireless video receiver that receives the video signal from the wireless video camera; and a PC video/USB adapter that plugs into the USB port of a PC computer and into the wireless video receiver. The adapter converts the video signal from the camera into a digital format for the PC computer. The PC computer at the remote location can then send this video to the central computer via the Internet.

The present invention allows a physician at a central office to treat multiple patients at multiple remote sites without the necessity of the patients coming to the central office for treatment. A respective computerized radiation treatment device at each remote location can be loaded remotely with a respective patient-specific digital data set comprising waveform parameters and treatment protocol data. The digital data set is transmitted from the central office to each remote site by any conventional means, e.g. downloading via the Internet; e-mail communication with attached file; telephone call; or physical delivery of a diskette or compact disk having the digital data set stored thereon. Each patient can self-administer a radiation treatment by donning a head coil antenna and turning the radiation treatment device on. The radiation treatment device will then cause the coil antenna to transmit electromagnetic radiation in accordance with the parameters and protocol contained in the patient-specific digital data set.

Optionally, a standard telephone line modem could be used in place of a DTMF transceiver.

Although the radiation treatment methods disclosed herein have wide application in the field of medicine, they are particularly useful in the treatment of neurological disorders such as Parkinson's disease. In the treatment of Parkinson's disease, the methods and apparatus for treating the patient with radiation are preferably used in conjunction with the administration, prior to radiation treatment, of a pharmacological-nutritional supplement. In particular, the method comprises the step of administering to the patient a chemical composition that increases serotonin transmission prior to irradiating the patient's brain.

While the invention has been described with reference to particular embodiments, it will be understood by those skilled in the art that various changes may be made and equivalents may be substituted for members thereof without departing from the scope of the invention. In addition, many modifications may be made to adapt a particular situation to the teachings of the invention without departing from the essential scope thereof. Therefore it is intended that the invention not be limited to the particular embodiment disclosed as the best mode contemplated for carrying out this invention, but that the invention will include all embodiments falling within the scope of the appended claims.

As used in the claims, the term “computer” means any one of a variety of electronic devices that are capable of accepting data and instructions, executing the instructions to process the data, and outputting the results of the processing step. Examples of types of devices within the scope of this definition include, but are not limited to, a microcontroller unit, a central processing unit, a PC computer, a computer programmed with server software, and a laptop computer.

1. A method of treating patients diagnosed as having a neurological disorder, comprising the following steps:
(a) programming a first computer to output digital data representing parameters for electromagnetic radiation, said parameters being designed to effect treatment of a neurological disorder;
(b) coupling a radiation treatment device to said first computer for receiving said digital data output;
(c) coupling an electromagnetic radiation transmitter to said radiation treatment device;
(d) positioning said electromagnetic radiation transmitter so that transmitted electromagnetic radiation enters the brain of a patient diagnosed as having said neurological disorder; and
(e) activating said electromagnetic radiation transmitter to transmit electromagnetic radiation as a function of at least digital data output.

2. The method as recited in claim 1, wherein said weak electromagnetic radiation comprises alternating current electromagnetic fields having intensities in the range of 1 to 100 picotesla flux density.

3. The method as recited in claim 2, wherein said weak electromagnetic radiation comprises alternating current electromagnetic fields having intensities with an average optimal range of 6.5 to 10 picotesla flux density.

4. The method as recited in claim 1, wherein the transmitted electromagnetic radiation is directed to the region of the pineal gland.

5. The method as recited in claim 1, further comprising the step of administering to the patient a chemical composition that increases serotonin transmission, said administering step being performed before said activating step.

6. The method as recited in claim 1, further comprising the steps of:
(e) monitoring a symptom of disease in said patient prior to said programming step; and
(f) determining said parameters as a function of the results of said monitoring step.

7. The method as recited in claim 6, wherein step (e) is performed on said patient in a central office, whereas said patient is located at a site remote from said central office when step (d) is performed.

8. The method as recited in claim 6, wherein steps (a) through (d) are performed at a site remote from a central office and step (f) is performed at said central office.

9. The method as recited in claim 6, wherein step (e) comprises the following steps:
the patient tries to draw a straight or circular line; and
digital data representing the line drawn by the patient is sent electronically from the remote site to the central office, and
wherein step (f) comprises the flowing steps:
printing or displaying an image corresponding to the transmitted electronic data representing the drawn line; and
the physician determines the degree of hand tremor in the patient based on inspection of the image.

10. The method as recited in claim 1, wherein said first computer is located at a remote site, and said programming step is performed by sending instructions from a second computer at a central location to said first computer at said remote site.

11. The method as recited in claim 10, wherein said instructions are sent via a telephone connection.

12. The method as recited in claim 10, wherein said instructions are sent from said second computer at said central location to a third computer at said remote site via an Internet connection and are then sent from said third computer to said first computer.

13. The method as recited in claim 6, wherein step (e) comprises the following steps:
(g) administering different signals to a patient's brain; and
(h) monitoring a symptom during each signal administration; and
wherein step (f) comprises the following steps:
(i) determining which signal produces the optimum response of said symptom to treatment; and
(j) determining said parameters as a function of the parameters of said signal that produced the optimal response.

14. The method as recited in claim 13, wherein said symptom is hand tremor.

15. The method as recited in Claim 12, wherein step (e) comprises sending a video signal of said patient during treatment, said video signal being sent from said third computer to said second computer.

16. A method of treating patients at sites remote from a central office, comprising the following steps:
(a) generating first through N-th digital data sets comprising waveform parameters and treatment protocol data, wherein N is a positive integer greater than unity;
(b) sending said first through N-th digital data sets from a central office to respective first through N-th remote locations; and
(c) treating respective first through N-th patients at said first through N-th remote locations, each treatment being a function of the respective one of said first through N-th digital data sets received at the respective remote location.

17. The method as recited in claim 16, wherein each treatment comprises transmitting electromagnetic radiation into the brain of the patient using a respective radiation treatment device, each of said first through N-th patients respectively receiving electromagnetic radiation having properties defined at least in part by said first through N-th digital data sets respectively.

18. The method as recited in claim 16, wherein said sending step comprises sending each of said first through N-th digital data sets directly to a respective treatment device located at said first through N-th remote locations via the public telephone switching network.

19. The method as recited in claim 16, wherein said sending step comprises sending each of said first through N-th digital data sets to a respective PC computer located at said first through N-th remote locations, and then loading said respective digital data set from said respective PC computer into a respective treatment device located at said first through N-th remote locations.

20. The method as recited in claim 16, wherein said sending step comprises loading said respective digital data sets into first through N-th treatment devices respectively at said central office and then physically transporting said treatment devices to said first through N-th remote locations.

21. The method as recited in claim 16, further comprising the steps of:
(d) monitoring from said central location a symptom of disease in one of said patients during said step of treating that patient at said respective remote location; and
(e) determining said waveform parameters of said digital data set for that patient as a function of the results of said monitoring step, said determining step being performed at said central location.

22. The method as recited in claim 21, wherein step (d) comprises sending a video signal of said patient during treatment, said video signal being sent from said respective remote location to said central location.

23. A system for treating patients having a neurological disorder, comprising:
a radiation treatment device comprising an antenna and circuitry for converting a digital data set into analog drive signals that are output to said antenna; and
a computer programmed to send said digital data set to said radiation treatment device via an electrical pathway.

24. The system as recited in claim 23, wherein said digital data set comprises waveform parameters and treatment protocol data.

25. The system as recited in claim 23, wherein said circuitry of said radiation treatment device comprises a programmable controller and a serial communications interface connected to said programmable controller, and said electrical pathway comprises a cable connected to said serial communications interface.

26. The system as recited in claim 23, wherein said circuitry of said radiation treatment device comprises:
a controller programmed to generate a digital waveform signal representing a waveform having properties that are a function of said digital data set received from said computer; and
a digital-to-analog converter for converting said digital waveform signal into an analog waveform signal,
wherein said antenna receives a signal derived from said analog waveform signal.

27. The system as recited in claim 26, further comprising a digital potentiometer connected to said controller and to said digital-to-analog converter.

28. The system as recited in claim 23, wherein said antenna is part of a headgear assembly.

29. A radiation treatment device comprising: an antenna; a controller programmed to output digital waveform signals that are a function of stored waveform parameters and treatment protocol data; and a digital-to-analog converter for converting said digital waveform signals into analog waveform signals, wherein said antenna is driven by drive signals derived from said analog waveform signals.

30. The device as recited in claim 29, further comprising: a DTMF transceiver connected to said controller; and a telephone line interface connected to said DTMF transceiver.

31. The device as recited in claim 30, further comprising a serial communications interface connected to said controller.

32. The device as recited in claim 29, further comprising: a battery; and a voltage stabilizer coupled to said controller and to said battery for stabilizing a voltage supplied to said controller by said battery.

33. The device as recited in claim 29, further comprising a digital potentiometer connected to said controller and to said digital-to-analog converter.

34. The device as recited in claim 29, wherein said antenna is part of a headgear assembly.

35. The device as recited in claim 29, further comprising a sensor coupled to said antenna and to said controller for providing a signal indicating improper connection of said antenna.

36. The device as recited in claim 29, further comprising an amplifier assembly connected to said antenna and to said digital-to-analog converter, wherein said controller comprises an input connected to an output of said amplifier assembly.

37. The device as recited in claim 29, wherein said controller is further programmed to count the number of treatments delivered and refuse to deliver additional treatments when the number of treatments delivered equals a predetermined threshold value stored in said controller.

38. A system for treating patients at sites remote from a central office, comprising:
a central treatment management computer programmed to generate first through N-th digital data sets comprising waveform parameters and treatment protocol data, wherein N is a positive integer greater than unity;
means for sending said first through N-th digital data sets from said central treatment management computer to respective first through N-th remote locations; and
means for treating respective first through N-th patients at said first through N-th remote locations, each treatment being a function of the respective one of said first through N-th digital data sets received at the respective remote location.

39. The system as recited in claim 38, wherein said treating means comprise first through N-th radiation treatment devices, each of said radiation treatment devices comprising an antenna and circuitry for driving said antenna to transmit electromagnetic radiation into the brain of the patient, each of said first through N-th patients respectively receiving electromagnetic radiation having properties defined at least in part by said first through N-th digital data sets respectively.

40. The system as recited in claim 39, wherein each of said first through N-th radiation treatment devices comprises a respective telephone line interface and a respective DTMF transceiver for receiving said first through N-th digital data sets respectively via the public telephone switching network.

41. The system as recited in claim 39, further comprising first through N-th PC computers respectively located at said first through N-th remote locations, each of said PC computers being programmed to receive a respective one of said first through N-th digital data sets from said central treatment management computer via the Internet and then load said respective digital data sets into a respective treatment device located at said first through N-th remote locations.

42. The system as recited in claim 39, wherein each of said first through N-th radiation treatment devices comprises an antenna and circuitry for converting a respective one of said first through N-th digital data sets into analog drive signals that are output to said antenna.

43. The system as recited in claim 42, wherein said circuit comprises: a controller programmed to output digital waveform signals that are a function of waveform parameters and treatment protocol data in a corresponding one of said digital data sets; and a digital-to-analog converter for converting said digital waveform signals into analog waveform signals, wherein said antenna is driven by drive signals derived from said analog waveform signals.

44. A method of treating patients having a neurological disorder, comprising the following steps:
(a) determining a digital data set that characterizes a radiation treatment for a patient having a neurological disorder;
(b) loading said digital data set into a radiation transmission controller; and
(c) transmitting radiation into a patient's brain under the control of said controller, said radiation having properties that are a function of said digital data set.

45. A system comprising a first computer programmed with parameter selection software, said parameter selection software comprising an operator interface for operator selection of parameters characterizing radiation for treating a patient diagnosed to have a neurological disorder and operator input of a load parameter command, and further comprising a routine for outputting a message comprising digital data representing said selected parameters in response to said operator input of said load parameter command.

46. The system as recited in claim 45, further comprising:
a second computer coupled to said first computer via an electrical pathway and programmed to output digital sample values representing radiation, said digital sample values being a function of said digital data; and
an assembly for transmitting radiation that is a function of at least said digital sample values.

47. The system as recited in claim 46, wherein said assembly comprises a head coil antenna designed to transmit radiation into a patient's brain.

48. The system as recited in claim 45, further comprising a display screen, wherein said operator interface comprises a graphical user interface, said graphical user interface comprising a first window for selecting an amplitude, a second window for selecting a frequency, and a third window for selecting a waveshape, said first through third windows being displayed concurrently or in sequence on said display screen.

49. The system as recited in claim 47, wherein said head coil antenna comprises an 8-shaped coil.
